SEQUENCE LISTING (6 sequences)

SEQ ID NO: 1 - DNA, 20 bp, synthetic construct
  source 1..20: mol_type = other DNA
tcgtgaacgaagacgaaagg

SEQ ID NO: 2 - DNA, 21 bp, synthetic construct
  source 1..21: mol_type = other DNA
tctgtgcctcctcaaagaatg

SEQ ID NO: 3 - DNA, 21 bp, synthetic construct
  source 1..21: mol_type = other DNA
ggccaacactccatgactcca

SEQ ID NO: 4 - DNA, 21 bp, synthetic construct
  source 1..21: mol_type = other DNA
tggggaatatyggaggcagag

SEQ ID NO: 5 - DNA, 21 bp, synthetic construct
  source 1..21: mol_type = other DNA
gcaccttaggccaacataaag

SEQ ID NO: 6 - DNA, 21 bp, synthetic construct
  source 1..21: mol_type = other DNA
ttgtctactccagcactcaaa

(19) United States
(12) Patent Application Publication
(10) Pub. No.: US 2025/0029679 A1
(43) Pub. Date: Jan. 23, 2025

(54) GENOTYPING OF CELL-FREE DNA FROM EMBRYO CULTURE MEDIA

(71) Applicant: VYTELLE LLC, Lenexa, KS (US)

(72) Inventors: Tasha Gruhot, Dublin, TX (US); Bruno Valente Sanches, Southlake, TX (US)

(21) Appl. No.: 18/756,080

(22) Filed: Jun. 27, 2024

Related U.S. Application Data
(60) Provisional application No. 63/510,448, filed on Jun. 27, 2023.

(51) Int. Cl. G16B 30/10; C12Q 1/6806; C12Q 1/686; C12Q 1/6881

(52) U.S. Cl. CPC G16B 30/10; C12Q 1/6806; C12Q 1/686; C12Q 1/6881

(57) ABSTRACT

The present disclosure provides, inter alia, techniques, technologies, and methods for non-invasive genotyping and/or embryo sexing, which methods include obtaining media containing cell-free DNA sluffed from an embryo, amplifying the cell-free DNA, and determining the genotype and/or sex of the embryo. In further aspects, the present disclosure provides methods for obtaining estimated breeding values from embryos non-invasively. Also provided are amplification processes to determine embryo genotype including sex.

### CROSS REFERENCE TO RELATED APPLICATIONS

This application claims priority under 35 U.S.C. § 119 to provisional patent application U.S. Ser. No. 63/510,448, filed Jun. 27, 2023. The provisional patent application is herein incorporated by reference in its entirety, including without limitation, the specification, claims, and abstract, as well as any figures, tables, appendices, or drawings thereof.

### SEQUENCE LISTING XML

The instant application contains a sequence listing, which has been submitted in XML file format by electronic submission and is hereby incorporated by reference in its entirety. The XML file, created on Oct. 9, 2024, is named P14325US01.xml and is 6,307 bytes in size.

### FIELD OF THE INVENTION

The present disclosure relates, generally, to technologies and methodologies for genotyping organisms and for improving both the predictive nature of such techniques, technologies, and methodologies while minimizing invasiveness and detrimental effects to embryos.

### BACKGROUND OF THE INVENTION

The background description provided herein gives context for the present disclosure. Work of the presently named inventors, as well as aspects of the description that may not otherwise qualify as prior art at the time of filing, are neither expressly nor impliedly admitted as prior art.

In vitro fertilization (IVF) and embryo transfer techniques are common practices within bovine calf production, and the processes maintain distinct advantages over traditional in vivo fertilization. First, the frequency in which IVF aspirations can be performed is much higher than traditional in vivo flushing techniques. While ovary aspirations for IVF can be performed every two weeks resulting in on average 6 embryos per aspiration, in vivo flushing produces five or six embryos every sixty days. Additionally, pregnant donor cows can still be utilized for oocyte collection as late as four months into pregnancy, furthering the throughput of embryo production.

Given the high throughput of calf production through IVF, the process can result in rapid genetic gain for the breeder through increased selection intensity and shortening generation interval. Additionally, genotyping the organism results in faster more accurate detection of high performing genetics, with significant implications on both a breeding impact and economic basis.

Current techniques for genotyping an embryo are limited. Prior to fertilization, semen sorting for sex per cell results in 85-90% accuracy of producing the desired sex embryo created from the sorted semen. However, that does not provide a basis for genotyping the resultant embryo. After fertilization, genotyping through embryo biopsy impacts embryo viability and is therefore infrequently utilized. Thus, there remains an unmet need in the art for technologies and methodologies resulting in highly accurate yet noninvasive genotyping of the embryo.

Thus, there exists a need in the art for a system and method for non-invasive genotyping of organisms.

### GLOSSARY

Unless defined otherwise, all technical and scientific terms used above have the same meaning as commonly understood by one of ordinary skill in the art to which embodiments of the present invention pertain.

The terms “a,” “an,” and “the” include both singular and plural referents.

The term “or” is synonymous with “and/or” and means any one member or combination of members of a particular list.

The terms “invention” or “present invention” are not intended to refer to any single embodiment of the particular invention but encompass all possible embodiments as described in the specification and the claims.

The term “about” as used herein refer to slight variations in numerical quantities with respect to any quantifiable variable. Inadvertent error can occur, for example, through use of typical measuring techniques or equipment or from differences in the manufacture, source, or purity of components.

The term “collection chamber” refers to a device for storing liquid such as a PCR tube, pipette, or other liquid holding apparatus.

The term “substantially” refers to a great or significant extent. “Substantially” can thus refer to a plurality, majority, and/or a supermajority of said quantifiable variable, given proper context.

The term “generally” encompasses both “about” and “substantially.”

The term “configured” describes structure capable of performing a task or adopting a particular configuration. The term “configured” can be used interchangeably with other similar phrases, such as constructed, arranged, adapted, manufactured, and the like.

Terms characterizing sequential order, a position, and/or an orientation are not limiting and are only referenced according to the views presented.

The “scope” of the present invention is defined by the appended claims, along with the full scope of equivalents to which such claims are entitled. The scope of the invention is further qualified as including any possible modification to any of the aspects and/or embodiments disclosed herein which would result in other embodiments, combinations, subcombinations, or the like that would be obvious to those skilled in the art.

Where a term is provided in the singular, other embodiments described by the plural of that term are also provided.

As used herein, the terms “include,” “includes,” and “including” are to be construed as at least having the features to which they refer while not excluding any additional unspecified features. It will be understood that, unless indicated to the contrary, terms intended to be “open” (e.g., the term “including” should be interpreted as “including but not limited to,” the term “having” should be interpreted as “having at least,” the term “includes” should be interpreted as “includes but is not limited to,” etc.). Phrases such as “at least one,” and “one or more,” and terms such as “a” or “an” include both the singular and the plural.

The term “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Terms characterizing sequential order, a position, and/or an orientation are not limiting and are only referenced according to the views presented.

### BRIEF DESCRIPTION OF THE DRAWINGS

The patent or application file contains at least one drawing executed in color. Copies of this patent or patent application publication with color drawing(s) will be provided by the Office upon request and payment of the necessary fee

Several embodiments in which the present invention can be practiced are illustrated and described in detail. The drawings are presented for exemplary purposes and may not be to scale unless otherwise indicated. FIGS. 1-8, 9A and 9B are pictures of the gel electrophoresis, therefore band intensity is not measured quantitatively.

FIG. 1 illustrates the binding of identified primers for the purposes of embryo sexing utilizing the SRY primer pair from somatic DNA (ear tissue samples).

FIG. 2 illustrates the binding of identified primers for the purposes of embryo sexing utilizing the AMEL primer pair from somatic DNA (ear tissue samples).

FIG. 3 illustrates assay functionality at 200 fg of DNA utilizing the SRY primer pair. The figure displays a successful binding of the primers to the DNA when provided with 200 fg of DNA.

FIG. 4 illustrates assay ladder functionality for DNA utilizing the SRY primer pair.

FIG. 5 illustrates assay functionality at 20 fg of DNA utilizing the SRY primer pair. The figure displays a successful binding of the primer to the DNA when provided with 20 fg of DNA.

FIG. 6 illustrates assay functionality at 200 fg of DNA utilizing the AMEL primer pair. The figure displays a successful binding of the primer to the DNA when provided with 200 fg of DNA.

FIG. 7 illustrates assay ladder functionality for DNA utilizing the AMEL primer pair.

FIG. 8 illustrates assay functionality at 20 fg of DNA utilizing the AMEL primer pair. The figure displays a successful binding of the primer to the DNA when provided with 20 fg of DNA.

FIG. 9A illustrates sex determination results of cell-free DNA from the SCM via touchdown PCR compared to the paired embryo sex.

FIG. 9B is an additional example that illustrates sex determination results of cell-free DNA in the SCM via touchdown PCR compared to the paired embryo.

FIG. 10 illustrates primer binding results from cell-free DNA from the SCM that underwent whole genome amplification.

FIG. 11 illustrates amplification and sex determination results across multiple replicates using the touchdown PCR method described in FIG. 10.

FIG. 12 illustrates the Next Gen Sequencing genetic concordance, genetic similarity and reads in common between embryos and the paired cell-free DNA of the SCM that the embryos were individually grown in.

FIG. 13 illustrates the percentage of reads sequenced that map to the cattle genome for biopsied embryos, cell-free DNA from SCM and from DNA contamination of the control media.

FIG. 14 illustrates future workflow that will use whole genome amplification followed by array based genotyping from the embryonic cell-free DNA.

### SUMMARY OF THE INVENTION

The following objects, features, advantages, aspects, and/or embodiments, are not exhaustive and do not limit the overall disclosure. No single embodiment needs to provide each and every object, feature, or advantage. Any of the objects, features, advantages, aspects, and/or embodiments disclosed herein can be integrated with one another, either in full or in part.

The present disclosure fulfills unmet needs in the art by providing technologies and methods for non-invasive genotyping of organisms. The technologies and methods disclosed herein employ collection of cell-free DNA which has been shed (sluffed) from an embryo into the media surrounding it, thus overcoming limitations in the art by providing non-invasive genotyping.

Within certain embodiments, provided herein are methods for determining the genotype of an embryo through collection of cell-free DNA within the culture media surrounding the embryo. In certain aspects, these methods comprise (1) collecting media surrounding an embryo, wherein said media contains cell-free DNA sluffed from the embryo, (2) performing touchdown PCR or whole genome amplification analysis on the cell-free DNA in the culture media utilizing optimized parameters and conditions to determination of the sex of an embryo (3) using DNA amplification methods which can include whole genome amplification to amplify the cell-free DNA for the purposes of producing genotype or sequence data for the embryo or to determine embryo sex (4) identifying contaminate DNA within the media and distinguishing said DNA from target embryo DNA (5) performing next generation sequencing (NGS), targeted sequencing, or array based genotyping utilizing optimized parameters and conditions to determine the sex and/or genotype of the embryo.

Within further embodiments, provided herein are the systems for using cell-free DNA analysis of embryo culture media for prenatal/embryonic genotyping, comprising (1) a collection device for collecting a sample of embryo culture media containing cell-free DNA, (2) an amplification unit for amplifying the cell-free DNA, (3) a unit for sequencing or genotyping the amplified cell-free DNA, (4) and a computer processor for comparing the DNA sequence to a reference genome to determine the genotype of the embryo.

Within still further embodiments, provided herein are the systems for using cell-free DNA analysis from embryo culture media for embryo sexing, comprising (1) a collection device for collecting a sample of embryo culture media containing cell-free DNA from a pregnant subject, (2) a purification unit for purifying the cell-free DNA from the sample, (3) an amplification unit for amplifying the purified cell-free DNA, (4) a unit for sequencing the amplified cell-free DNA, (5) and a computer processor for comparing the DNA sequence to a reference genome to determine the sex of the embryo.

Within still further embodiments, provided herein are the systems for using cell-free DNA analysis from embryo culture media for embryo sexing, comprising (1) a collection device for collecting a sample of embryo culture media containing cell-free DNA from a pregnant subject, (2) an amplification unit for amplifying the cell-free DNA, (3) a PCR based assay for determining embryo sex from the cell-free DNA (4) a unit for sequencing the amplified cell-free DNA, (5) and a computer processor for comparing the DNA sequence to a reference genome to determine the sex of the embryo.

Within still further embodiments, provided herein are any devices for genotyping an organism through cell-free DNA analysis from embryo culture media, comprising: a collection chamber for collecting a sample of embryo culture media containing cell-free DNA; a purification unit for purifying the cell-free DNA from the sample; an amplification method for amplifying the cell-free DNA; and a unit for sequencing or genotyping the cell-free DNA to determine the genotype of the organism.

Within still further embodiments, provided herein are any devices for embryonic sexing through cell-free DNA analysis from embryo culture media, comprising: a collection chamber for collecting a sample of embryo culture media containing cell-free DNA; an amplification method for amplifying the cell-free DNA; and a sequencing and/or genotyping method for sequencing/genotyping the cell-free DNA to determine the sex of the embryo.

Within still further embodiments, provided herein are any devices for embryonic sexing through cell-free DNA analysis from embryo culture media, comprising: a collection chamber for collecting a sample of embryo culture media containing cell-free DNA; an amplification method for amplifying the cell-free DNA; and a PCR based assay for determining embryo sex from the cell-free DNA.

Within still further embodiments, provided herein are any kits for genotyping an organism through cell-free DNA analysis from embryo culture media, comprising (1) a collection device for collecting a sample of embryo culture media containing cell-free DNA, (2) optionally, a purification unit for purifying the cell-free DNA from the sample, (3) an amplification method for amplifying the cell-free DNA (4) a sequencing method for sequencing or genotyping the purified cell-free DNA, (4) and a computer program for comparing the DNA sequence to a reference genome to determine the genotype of the organism.

Within still further embodiments, provided herein are any kits for sexing an embryo through cell-free DNA analysis from embryo culture media, comprising (1) a collection device for collecting a sample of embryo culture media containing cell-free DNA, (2) an amplification method for amplifying the cell-free DNA (3) a method for sequencing or genotyping the cell-free DNA, (4) and a computer program for comparing the DNA sequence to a reference genome to determine the sex of the embryo.

Within still further embodiments, provided herein are any kits for sexing an embryo through cell-free DNA analysis from embryo culture media, comprising (1) a collection device for collecting a sample of embryo culture media containing cell-free DNA, (2) a method for performing PCR directly from the media, and (3) and an assay to determine sex specific primer amplification to determine the sex of the embryo.

Within still further embodiments, provided herein is a computer-implemented system for genotyping an organism through cell-free DNA analysis from embryo culture media, comprising: a collection device for collecting a sample of embryo culture media containing cell-free DNA; an amplification method for amplifying the cell-free DNA; a method for sequencing and/orgenotyping the purified cell-free DNA; and a computer processor for comparing and/or imputing the sequence/genotype data to a reference genome to determine the genotype of the organism.

Within still further embodiments, provided herein is a computer-implemented system for embryonic sexing through cell-free DNA analysis from embryo culture media, comprising: a collection device for collecting a sample of embryo culture media containing cell-free DNA; an amplification method for amplifying the cell-free DNA; a method for sequencing and/or genotyping the purified cell-free DNA; and a computer processor for comparing and/or imputing the sequence/genotype data to a reference genome to determine the sex of the embryo.

These and other related aspects of the present disclosure will be better understood in light of the following drawings and detailed description, which exemplify various embodiments, and which are presented for illustration, not limitation.

### EMBODIMENTS

Various embodiments of the systems and methods provided herein are included in the following non-limiting list of embodiments.

1. A method for genotyping an organism through cell-free DNA analysis from embryo culture media, comprising the steps of: collecting a sample of embryo culture media containing cell-free DNA; amplifying the cell-free DNA from the sample; and sequencing the amplified cell-free DNA to determine the genotype of the organism.

2. A device for genotyping an organism through cell-free DNA analysis from embryo culture media, comprising: a collection chamber for collecting a sample of embryo culture media containing cell-free DNA; an amplification unit for amplifying the cell-free DNA from the sample; and a sequencing unit for sequencing the cell-free DNA to determine the genotype of the organism.

3. A system for genotyping an organism through cell-free DNA analysis from embryo culture media, comprising: a collection device for collecting a sample of embryo culture media containing cell-free DNA; an amplification unit for amplifying the cell-free DNA from the sample; and a sequencing unit for sequencing the cell-free DNA to determine the genotype of the organism; wherein the system is capable of generating a genomic profile of the organism.

4. A computer-implemented method for genotyping an organism through cell-free DNA analysis from embryo culture media, comprising the steps of: collecting a sample of embryo culture media containing cell-free DNA; amplifying the cell-free DNA from the sample; sequencing the cell-free DNA; and comparing the sequence data to a reference genome to determine the genotype of the organism.

5. A device for genotyping an organism through cell-free DNA analysis from embryo culture media, comprising: a collection chamber for collecting a sample of embryo culture media containing cell-free DNA; an amplification unit for amplifying the cell-free DNA from the sample; a sequencing unit for sequencing the cell-free DNA; and a computer processor for comparing the sequence data to a reference genome to determine the genotype of the organism.

6. A kit for genotyping an organism through cell-free DNA analysis from embryo culture media, comprising: a collection device for collecting a sample of embryo culture media containing cell-free DNA; an amplification unit for amplifying the cell-free DNA from the sample; a sequencing unit for sequencing the cell-free DNA; and a computer program for comparing the sequence data to a reference genome to determine the genotype of the organism.

7. A method for non-invasive genotyping of an organism through cell-free DNA analysis from embryo culture media, comprising the steps of: collecting a sample of embryo culture media containing cell-free DNA; amplifying the cell-free DNA from the sample; sequencing the amplified cell-free DNA; and comparing the sequence data to a reference genome to determine the genotype of the organism without causing harm to the organism.

8. A device for non-invasive genotyping of an organism through cell-free DNA analysis from embryo culture media, comprising: a collection chamber for collecting a sample of embryo culture media containing cell-free DNA; an amplification unit for amplifying the cell-free DNA from the sample; a sequencing unit for sequencing the cell-free DNA; and a computer processor for comparing the sequence data to a reference genome to determine the genotype of the organism without causing harm to the organism.

9. A computer-implemented system for genotyping an organism through cell-free DNA analysis from embryo culture media, comprising: a collection device for collecting a sample of embryo culture media containing cell-free DNA; an amplification unit for amplifying the cell-free DNA from the sample; a sequencing unit for sequencing the cell-free DNA; and a computer processor for comparing the DNA sequence to a reference genome to determine the genotype of the organism.

10. A method for genotyping an organism through cell-free DNA analysis from embryo culture media, comprising the steps of: collecting a sample of embryo culture media containing cell-free DNA from a subject; purifying the cell-free DNA from the sample; amplifying the cell-free DNA; sequencing the cell-free DNA to determine the genotype of the organism.

11. A device for genotyping an organism through cell-free DNA analysis from embryo culture media, comprising: a collection chamber for collecting a sample of embryo culture media containing cell-free DNA; a purification unit for purifying the cell-free DNA from the sample; an amplification unit for amplifying the cell-free DNA; and a sequencing unit for sequencing the cell-free DNA to determine the genotype of the organism.

12. A system for genotyping an organism through cell-free DNA analysis from embryo culture media, comprising: a collection device for collecting a sample of embryo culture media containing cell-free DNA; a purification unit for purifying the cell-free DNA from the sample; an amplification unit for amplifying the purified cell-free DNA; a sequencing unit for sequencing the cell-free DNA; and a computer processor for comparing the DNA sequence to a reference genome to determine the genotype of the organism.

13. A method for genotyping an organism through cell-free DNA analysis from embryo culture media, comprising the steps of: collecting a sample of embryo culture media containing cell-free DNA from a subject; amplifying the cell-free DNA from the sample; sequencing the amplified cell-free DNA; and comparing the DNA sequence to a reference genome to determine the genotype of the organism.

14. A device for genotyping an organism through cell-free DNA analysis from embryo culture media, comprising: a collection chamber for collecting a sample of embryo culture media containing cell-free DNA; an amplification unit for amplifying the cell-free DNA; a sequencing unit for sequencing the amplified cell-free DNA; and a computer processor for comparing the sequence data to a reference genome to determine the genotype of the organism.

15. A system for non-invasive genotyping of an organism through cell-free DNA analysis from embryo culture media, comprising: a collection device for collecting a sample of embryo culture media containing cell-free DNA from a subject; an amplification unit for amplifying the purified cell-free DNA; a sequencing unit for sequencing the amplified cell-free DNA; and a computer processor for comparing the DNA sequence to a reference genome to determine the genotype of the organism.

16. A method for using cell-free DNA analysis from embryo culture media for embryo genotyping, comprising the steps of: collecting a sample of embryo culture media containing cell-free DNA; amplifying the cell-free DNA; sequencing the amplified cell-free DNA; and comparing the DNA sequence to a reference genome to determine the genotype of the embryo.

17. A device for using cell-free DNA analysis from embryo culture media for prenatal genotyping, comprising: a collection chamber for collecting a sample of embryo culture media containing cell-free DNA; an amplification unit for amplifying the cell-free DNA; a sequencing unit for sequencing the amplified cell-free DNA; and a computer processor for comparing the sequence data to a reference genome to determine the genotype of the embryo.

18. A system for using cell-free DNA analysis from embryo culture media for prenatal genotyping, comprising: a collection device for collecting a sample of embryo culture media containing cell-free DNA; an amplification unit for amplifying the cell-free DNA; a sequencing unit for sequencing the amplified cell-free DNA; and a computer processor for comparing the sequence data to a reference genome to determine the genotype of the embryo.

19. A method for using cell-free DNA analysis from embryo culture media for prenatal genotyping, comprising the steps of: collecting a sample of embryo culture media containing cell-free DNA from a fertilized egg; amplifying the cell-free DNA; sequencing the amplified cell-free DNA; and comparing the sequence data to a reference genome to determine the genotype of the embryo.

20. A device for using cell-free DNA analysis from embryo culture media for prenatal genotyping, comprising: a collection chamber for collecting a sample of embryo culture media containing cell-free DNA from a fertilized egg; an amplification unit for amplifying the cell-free DNA; a sequencing unit for sequencing the amplified cell-free DNA; and a computer processor for comparing the sequence data to a reference genome to determine the genotype of the embryo.

21. A system for using cell-free DNA analysis from embryo culture media for prenatal genotyping, comprising: a collection device for collecting a sample of embryo culture media containing cell-free DNA from a fertilized egg; an amplification unit for amplifying the cell-free DNA; a sequencing unit for sequencing the amplified cell-free DNA; and a computer processor for comparing the sequence data to a reference genome to determine the genotype of the embryo.

22. The method of any of embodiments 1,7, 10, 13, 16, or 19 wherein the genotyping determines the sex of the organism.

23. The method of any of embodiments 4, 5, 7, 13, 16, or 19 wherein the method further comprises imputing the DNA sequence data.

24. A method for non-invasive embryo sexing of an organism through cell-free DNA analysis from embryo culture media, comprising the steps of: collecting a sample of embryo culture media containing cell-free DNA; amplifying the cell-free DNA from the sample; utilizing sex specific primers through PCR to bind to the cell-free DNA in the SCM, and determining the sex of the embryo.

25. A device for non-invasive embryo sexing of an organism through cell-free DNA analysis from embryo culture media, comprising: a collection chamber for collecting a sample of embryo culture media containing cell-free DNA; an amplification unit for amplifying the cell-free DNA from the sample utilizing sex specific primers through PCR to bind to the cell-free DNA in the SCM; and a computer processor to assess cell free-DNA amplification and primer binding to determine the sex of the embryo through the SCM.

26. A computer-implemented system for non-invasive embryo sexing of an organism through cell-free DNA analysis from embryo culture media, comprising: a collection chamber for collecting a sample of embryo culture media containing cell-free DNA; an amplification unit for amplifying the cell-free DNA from the sample utilizing sex specific primers through PCR to bind to the cell-free DNA in the SCM; and a computer processor to assess cell free-DNA amplification and primer binding to determine the sex of the embryo through the SCM.

27. A system for non-invasive embryo sexing of an organism through cell-free DNA analysis from embryo culture media, comprising the steps of: collecting a sample of embryo culture media containing cell-free DNA; amplifying the cell-free DNA from the sample; utilizing sex specific primers through PCR to bind to the cell-free DNA in the SCM, and determining the sex of the embryo.

While various embodiments have been disclosed herein, other embodiments will be apparent to those skilled in the art. The various embodiments disclosed herein are for purposes of illustration and are not intended to be limiting, with the true scope and spirit being indicated by the claims. Moreover, the embodiments described in this disclosure can be combined in various ways. Any aspect or feature that is described for one embodiment can be incorporated into any other embodiment mentioned in this disclosure. While various aspects of presently disclosed embodiments are described herein, it will be understood that various omissions and substitutions and changes may be made by those skilled in the art without departing from the spirit of this disclosure. Those skilled in the art will appreciate that the inventive principles can be practiced in other than the described embodiments, which are presented for purposes of illustration and not limitation. The disclosures of all patent and scientific literature cited herein are expressly incorporated by reference in their entirety.

### DETAILED DESCRIPTION OF THE INVENTION

The present disclosure is not to be limited to that described herein. Mechanical, electrical, chemical, procedural, and/or other changes can be made without departing from the spirit and scope of the present invention. No features shown or described are essential to permit the basic operation of the present invention unless otherwise indicated.

The present disclosure provides technologies, methods, devices, kits, and/or computer implemented systems for (1) isolating the embryo to allow for collection of spend culture media (SCM) (2) performing touchdown PCR with designated primer pairs or whole genome amplification analysis utilizing optimized parameters and conditions to determine the sex of the paired embryo to the cell-free DNA found in the SCM (4) performing cell-free DNA amplification from the SCM, followed by NGS, targeted sequencing, or array based genotyping utilizing optimized parameters and conditions to non-invasively determine the sex and/or genotype of the embryo.

Genotype imputation is an important tool in the analysis of sequence data and genomewide association scans. The technique allows geneticists to accurately evaluate the evidence for association at genetic markers that are not directly genotyped. Imputation methods are well known to those of skill in the art, such as noted in Annu. Rev. Genomics Hum. Genet. 2009; 10:387-406, hereby incorporated by reference in its entirety.

According to some embodiments, the present disclosure provides methods for genotyping an organism through cell-free DNA analysis from embryo culture media, comprising the steps of: collecting a sample of embryo culture media containing cell-free DNA; amplifying and sequencing and/or genotyping the amplified cell-free DNA to determine the genotype of the organism.

In related embodiments, the present disclosure provides methods for embryo sexing through cell-free DNA analysis from embryo culture media, comprising the steps of: collecting a sample of embryo culture media containing cell-free DNA; amplifying the cell-free DNA from the sample; and sequencing and/or genotyping the amplified cell-free DNA to determine the sex of the embryo.

In related embodiments, the genotyping step comprises sequencing only.

In related embodiments, the present disclosure provides methods for embryo sexing and/or genotyping through cell-free DNA analysis from embryo culture media, comprising the steps of: collecting a sample of embryo culture media containing cell-free DNA; performing touchdown PCR; and/or sequencing (and/or genotyping) the amplified cell-free DNA to determine the genotype of the embryo optionally including the sex of the embryo.

In other embodiments, the present disclosure provides for a device for genotyping an organism through cell-free DNA analysis from embryo culture media, comprising: a collection chamber for collecting a sample of embryo culture media containing cell-free DNA; an amplification unit for amplifying the cell-free DNA from the sample and a sequencing/genotyping unit for sequencing and or genotyping cell-free DNA to determine the genotype of the organism.

In related embodiments, the present disclosure provides for a device for embryo sexing through cell-free DNA analysis from embryo culture media, comprising: a collection chamber for collecting a sample of embryo culture media containing cell-free DNA; an amplification unit for amplifying the cell-free DNA from the sample; and a sequencing/genotyping unit for sequencing and or genotyping the amplified cell-free DNA to determine the sex of the embryo.

In still other embodiments, the present disclosure provides for a system for genotyping an organism through cell-free DNA analysis from embryo culture media, comprising: a collection device for collecting a sample of embryo culture media containing cell-free DNA; a amplification method for amplifying the cell-free DNA from the sample; and a sequencing/genotyping method for sequencing and or genotyping the amplified cell-free DNA to determine the genotype of the organism; wherein the system is capable of generating a genetic profile of the organism.

In related embodiments, the present disclosure provides for a system for embryo sexing through cell-free DNA analysis from embryo culture media, comprising: a collection device for collecting a sample of embryo culture media containing cell-free DNA; a amplification method for amplifying the cell-free DNA from the sample; and a sequencing/genotyping method for sequencing and or genotyping the amplified cell-free DNA to determine the sex of the embryo.

In even further embodiments, the present disclosure provides for a kit for genotyping an organism through cell-free DNA analysis from embryo culture media, comprising: a collection device for collecting a sample of embryo culture media containing cell-free DNA; an amplification unit for amplifying the cell-free DNA from the sample; and a sequencing/genotyping unit for sequencing and or genotyping the amplified cell-free DNA; and a computer program for comparing the DNA sequence to a reference genome to determine the genotype of the organism.

In related embodiments, the present disclosure provides for a kit for embryo sexing through cell-free DNA analysis from embryo culture media, comprising: a collection device for collecting a sample of embryo culture media containing cell-free DNA; an amplification unit for amplifying the cell-free DNA from the sample a sequencing/genotyping unit for sequencing and or genotyping the amplified cell-free DNA; and a computer program for comparing and/or imputing the sequence/genotype data to a reference genome to determine the sex of the embryo.

In even further embodiments, the present disclosure provides for a computer-implemented system for genotyping an organism through cell-free DNA analysis from embryo culture media, comprising: a collection device for collecting a sample of embryo culture media containing cell-free DNA; an amplification unit for amplifying the cell-free DNA from the sample a sequencing and/or genotyping unit for sequencing/genotyping the amplified cell-free DNA; and a computer processor for comparing and/or imputing the sequence/genotype data to a reference genome to determine the genotype of the organism.

In related embodiments, the present disclosure provides for a computer-implemented system for determining the sex of an embryo through cell-free DNA analysis from embryo culture media, comprising: a collection device for collecting a sample of embryo culture media containing cell-free DNA; an amplification unit for amplifying the cell-free DNA from the sample a sequencing/genotyping unit for sequencing and or genotyping the amplified cell-free DNA; and a computer processor for comparing and/or imputing the sequence/genotype data to a reference genome to determine the sex of the embryo.

Touchdown PCR is a method for increasing the specificity of PCR reactions. Cycling programs are used where the annealing temperature starts high and gradually reduces over the PCR cycles until the optimum annealing temperature is reached. Touchdown PCR overcomes issues such as primer dimer formation and non-specific amplification.

The “scope” of the present disclosure is defined by the appended claims, along with the full scope of equivalents to which such claims are entitled. The scope of the invention is further qualified as including any possible modification to any of the aspects and/or embodiments disclosed herein which would result in other embodiments, combinations, subcombinations, or the like that would be obvious to those skilled in the art.

It will be further understood that where features or aspects of the disclosure are described in terms of Markush groups, the disclosure is also intended to be described in terms of any individual member or subgroup of members of the Markush group. Similarly, all ranges disclosed herein also encompass all possible sub-ranges and combinations of sub-ranges and that language such as “between,” “up to,” “at least,” “greater than,” “less than,” and the like include the number recited in the range and includes each individual member.

All references cited herein, whether supra or infra, including, but not limited to, patents, patent applications, and patent publications, whether U.S., PCT, or non-U.S. foreign, and all technical and/or scientific publications are hereby incorporated by reference in their entirety.

### EXAMPLES

The present disclosure is further described with reference to the following examples, which are provided to illustrate certain embodiments and are not intended to limit the scope of the present disclosure or the subject matter claimed.

### Example 1

Analysis and Identification of Primer Pairs for target X and Y-Chromosome Regions and Analysis of X and Y-Chromosome Specific POR

This Example demonstrates that successful primer pairs for the amplification of target regions in both the male and female chromosomes were identified. Further, the accuracy of sex determination of an embryo based on its spent culture media (SCM) and optimal amount of culture media for PCR reactions were determined.

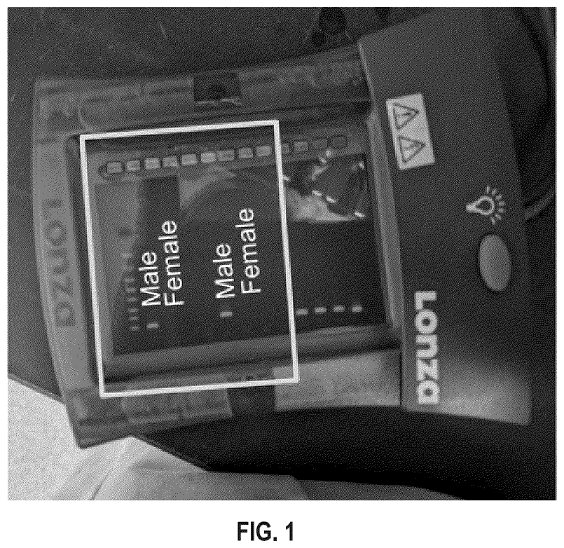

Results (methods) description: The following primer pairs were used to amplify specific DNA fragments: SRY FWD (SEQ ID NO: 1), SRY REV (SEQ ID NO: 2), AMEL FWD (SEQ ID NO: 3), AMEL REV (SEQ ID NO: 4), Par region on TBL1 FWD (SEQ ID NO: 5), Par region on TBL1 REV (SEQ ID NO: 6). Optimization and validation of these primers was conducted through PCR and touchdown PCR. Additionally, PCR and touchdown PCR assays were conducted to determine the sensitivity of quantification of DNA amounts from the SCM, as well additional validation experimentation. FIGS. 1-8, 9A and 9B are pictures of the gel electrophoresis, therefore band intensity is not measured quantitatively.

FIG. 1 illustrates the binding of identified primer pair SRY FWD (SEQ ID NO: 1). SRY REV (SEQ ID NO: 2), to the SRY gene using somatic DNA (ear tissue samples).

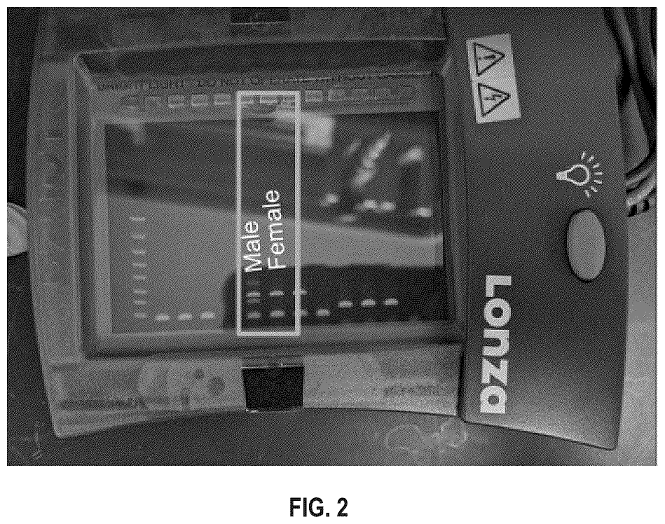

FIG. 2 illustrates the binding of identified primer pair to the AMEL FWD (SEQ ID NO: 3), AMEL REV (SEQ ID NO: 4) using somatic DNA (ear tissue samples).

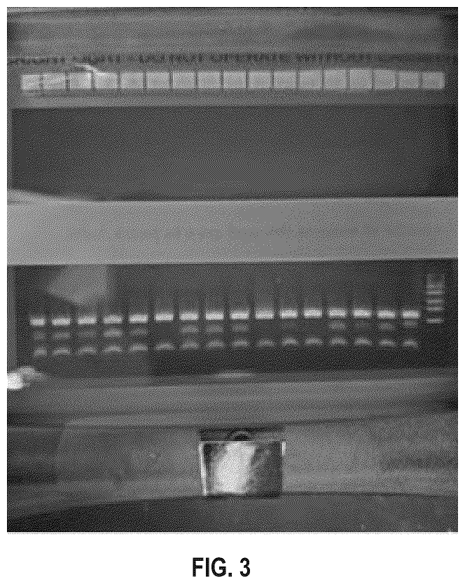

FIG. 3 illustrates assay functionality at 200 fg of DNA utilizing the SRY FWD (SEQ ID NO: 1), SRY REV (SEQ ID NO: 2) primer pair. The figure displays a successful binding of the primer to the DNA when provided with 200 fg of DNA.

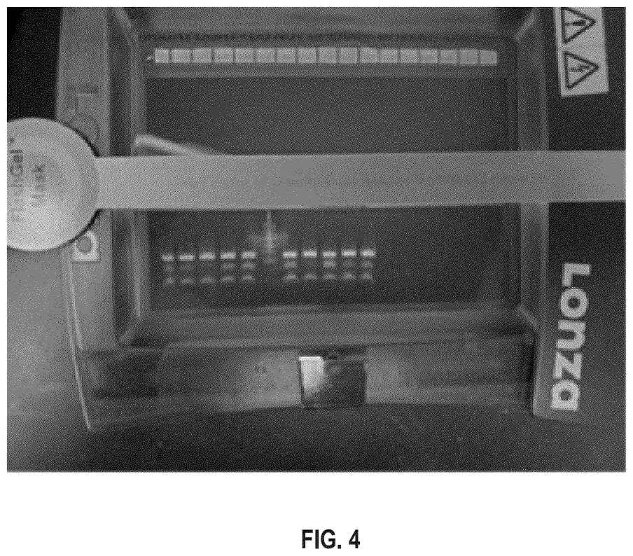

FIG. 4 illustrates assay ladder functionality for DNA utilizing the primer pair SRY FWD (SEQ ID NO: D), SRY REV (SEQ ID NO: 2).

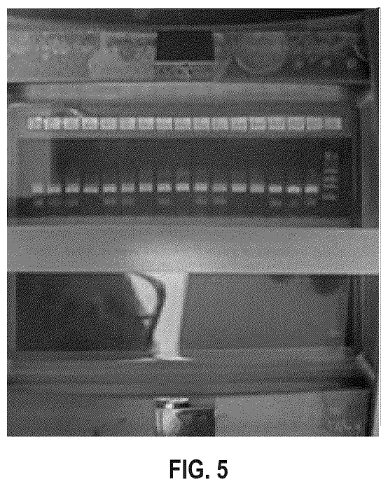

FIG. 5 illustrates assay functionality at 20 fg of DNA utilizing the SRY FWD (SEQ ID NO: 1), SRY REV (SEQ ID NO: 2) primer pair. The figure displays a successful binding of the primer to the DNA when provided with 20 fg of DNA.

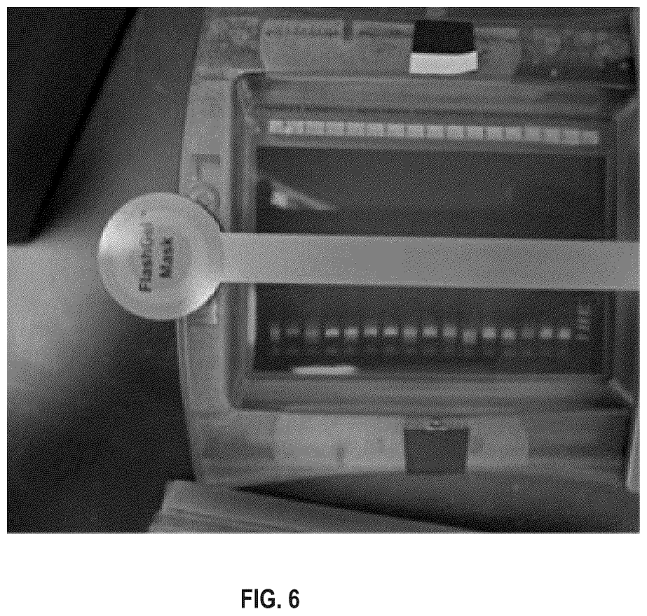

FIG. 6 illustrates assay functionality at 200 fg of utilizing the Par region on TBL1 FWD (SEQ ID NO: 5), Par region on TBL1 REV (SEQ ID NO: 6) primer pair. The figure displays a successful binding of the primer to the DNA when provided with 200 fg of DNA.

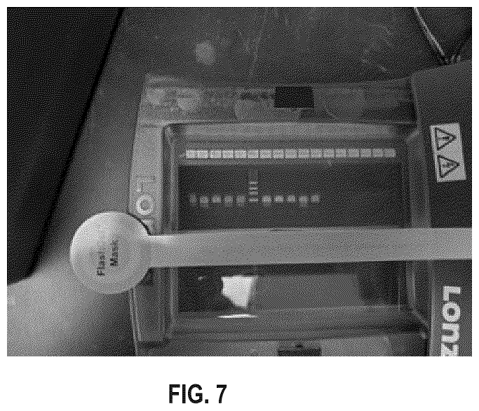

FIG. 7 illustrates assay ladder functionality for utilizing the Par region on TBL1 FWD (SEQ ID NO: 5), Par region on TBL1 REV (SEQ ID NO: 6) primer pair.

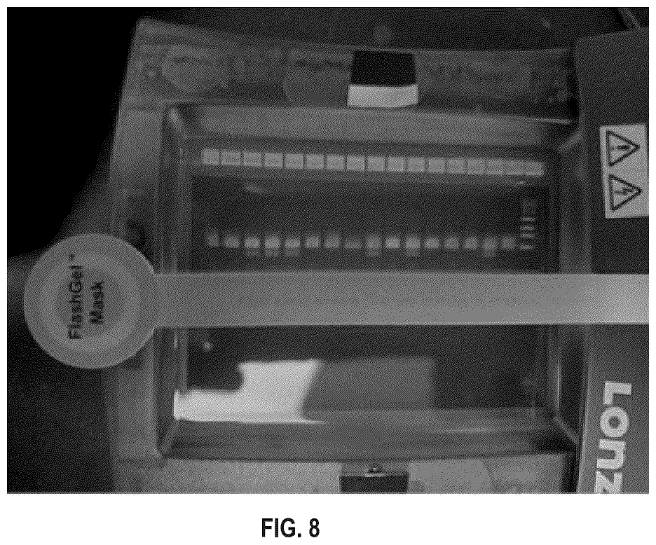

FIG. 8 illustrates assay functionality at 20 fg of utilizing the Par region on TBL1 FWD (SEQ ID NO: 5), Par region on TBL1 REV (SEQ ID NO: 6) primer pair. The figure displays a successful binding of the primer to the DNA when provided with 20 fg of DNA.

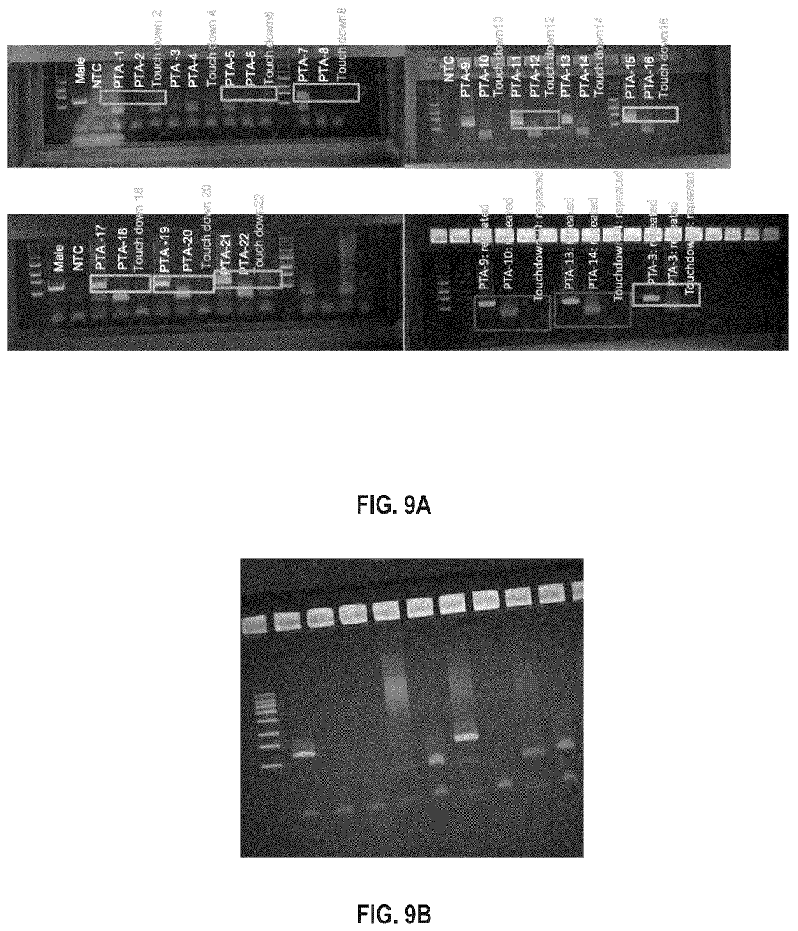

FIG. 9A illustrates an example of sex determination results of cell-free DNA in the SCM via touchdown PCR compared to the paired embryo sex.

Embryos were individually cultured from Day 4-Day 7. On Day 7, the embryo was removed from the culture media it was grown in since day 4 “spent culture media” (SCM). Both the embryo and the SCM (˜12 μL) were frozen.

Primers were utilized to determine sex:

SRY FWD (SEQ ID NO: 1), SRY REV (SEQ ID NO: 2), AMEL FWD (SEQ ID NO: 3), AMEL REV (SEQ ID NO: 4), Par region on TBL1 FWD (SEQ ID NO: 5), Par region on TBL1 REV (SEQ ID NO: 6).

The green boxes represent correct sex determination between the embryo and the paired SCM. The tan boxes represent correct sex determination between the embryo and the paired SCM but with weak band strength. The red boxes represent complete misses, where touchdown PCR failed.

Touchdown PCR: 9 out of 11 SCM samples were determined to be the same sex as the embryo that was grown in the media. Results indicate 0 false positives. 6 out of 11 fully capture and are concordant for sex, with 3 out of 11 exhibiting a weak signal but concordant for sex, and 2 out of 11 were complete misses.

Example: PTA-1 is the embryo for the paired SCM of PTA-2 and so on. Correct sex identification is highlighted by a green box, correct sex identification but with weak band strength is identified by a tan box, and a complete miss where touchdown PCR failed is highlighted by a red box. Figures are pictures of the gel electrophoresis, therefore band intensity is not measured quantitatively.

FIG. 9B is an additional example that illustrates sex determination results of cell-free DNA in the SCM via touchdown PCR compared to the paired embryo. The columns on the gel represent the ladder, male control, female control, NTC, embryo 1, embryo 1 SCM, Embryo 2, embryo 2 SCM, and embryo 3, embryo 3 SCM. This gel illustrates that both embryo 1 and 3 were sexed female via embryo biopsy and by the cell-free DNA touch down PCR assay of the SCM. Embryo 2 was sexed male via embryo biopsy and the cell-free DNA failed to amplify for the SCM in this example.

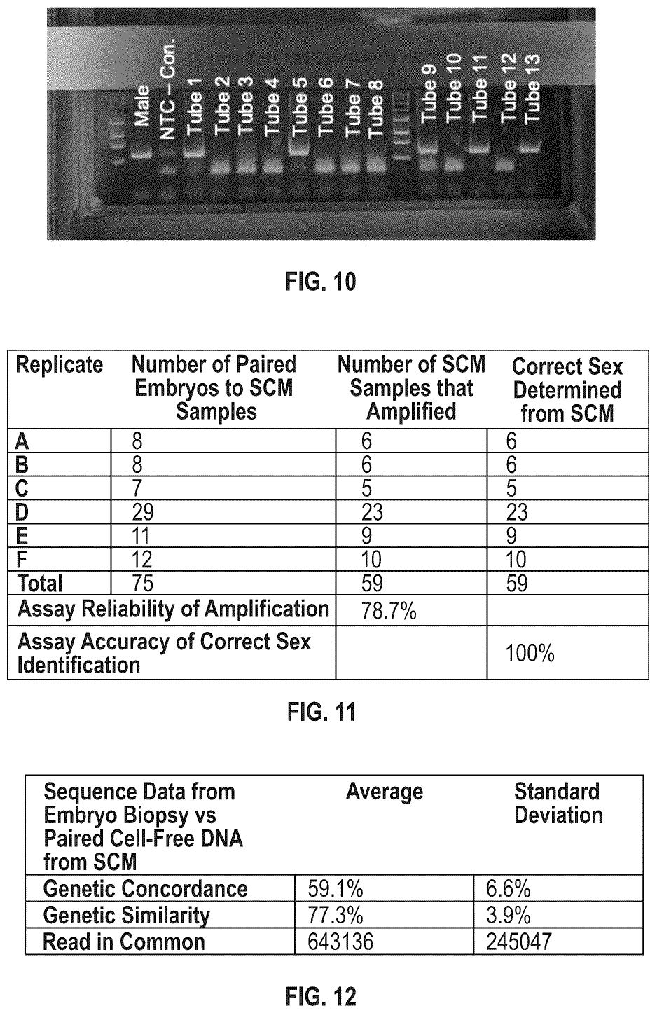

FIG. 10 illustrates results from WGA to determine sex via SCM.

DNA from the SCM was successfully whole genome amplified using the “ResolveDNA” platform (Bioskryb Genomics). However, for the purposes of determining embryo sex WGA did not improve results from touchdown PCR. Tube 1 is the embryo paired to the SCM in tube 2 and this patten continues on the gel.

FIG. 11 illustrates amplification and sex determination results across multiple replicates using the touchdown PCR method described in FIG. 10. The assay reliability of amplifying SCM cell-free DNA across all replicates is 79%, while the assay accuracy of determining the correct embryonic sex from the amplified SCM cell-free DNA is 100% across all replicates.

| | Number of paired | Number of SCM | Correct sex |
|---|---|---|---|
| | embryos | samples that | determined from |
| Replicate | to SCM samples | amplified | SCM |
| A | 8 | 6 | 6 |
| B | 8 | 6 | 6 |
| C | 7 | 5 | 5 |
| D | 29 | 23 | 23 |
| E | 11 | 9 | 9 |
| F | 12 | 10 | 10 |
| Total | 75 | 59 | 59 |

| | | |
|---|---|---|
| Assay reliability of amplification | 78.7% | |
| Assay accuracy of correct sex | | 100% |
| identification | | |

### Example 2

### The Use of Embryonic Cell-Free DNA for the Purposes of Sequencing or Genotyping the Embryo

FIG. 12 illustrates preliminary results of sequencing the cell-free DNA from SCM and the paired embryos. Genetic concordance, genetic similarity and reads in common are reported below for DNA biopsied from the embryos and the paired cell-free DNA of the media that the embryos were individually grown in. DNA was extracted using a commercially available Nucleic Acid extraction kit and sequencing was performed using Illumina platform Next Generation Sequencing. This set was composed of DNA collected from 28 embryos alongside the cell-free DNA from the paired SCM media. The average call rate across the cell-free SCM was 63.9%.

| Sequence data from Embryo | | |
|---|---|---|
| Biopsy vs paired Cell-Free DNA | | |
| from SCM | Average | Standard Deviation |
| Genetic Concordance | 59.1% | 6.6% |
| Genetic Similarity | 77.3% | 3.9% |
| Read in common | 643136 | 245047 |

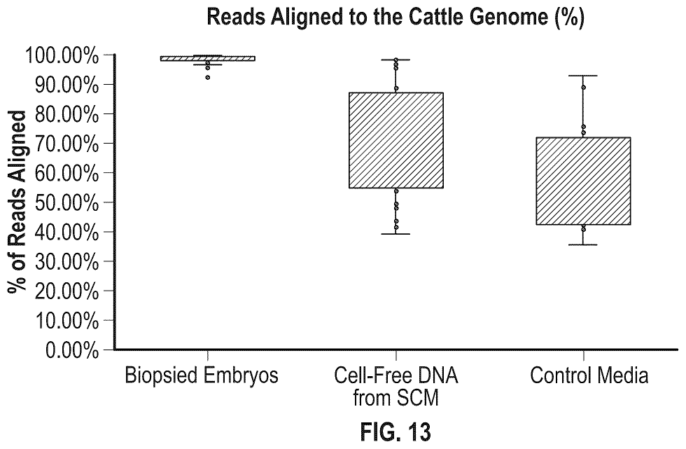

FIG. 13 illustrates the percentage of reads sequenced (in reference to FIG. 12) that map to the cattle genome for biopsied embryos, cell-free DNA from SCM and from DNA contamination of the control media. Evidence of DNA contamination occurred. This DNA contamination is occurring in the media prior to use and additionally may also be occurring during processing of the embryos and samples. 70% of the reads from the SCM aligned to the cattle genome. Clean control media that had never contained an embryo was also sequenced to test for DNA contamination, from these samples 56% of the reads aligned to the cattle genome. By using next generation sequencing (NGS) and targeted sequencing, bovine DNA contaminates in the control culture media (never exposed to an embryo) can be identified and used as a reference to compare to the SCM cell-free DNA sequence results allowing for the identification of embryonic originated DNA in the SCM versus contaminate. The identified embryonic DNA sequence data can then be used to determine the sex of the embryo as well as be utilized in breeding evaluations to in turn produce estimated breeding values for phenotypic traits of importance for said embryo.

### Specifics:

Qiagen QIAamp Circulating Nucleic Acid kit

Illumina, 2×150 bp, ˜350M PE reads (˜105 GB), dual index

Estimated output: ˜350<raw paired end reads per unit

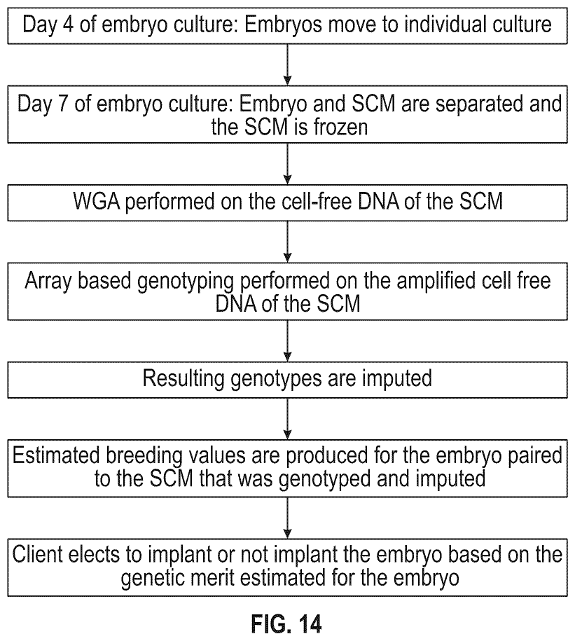

FIG. 14 illustrates future workflow that will use whole genome amplification followed by array based genotyping from the SCM cell-free DNA. The resulting genotypes will then be utilized to produce breeding values for the embryos on economically important traits which can be used for selection of embryo implantation. The following work flow will be utilized:

Embryos will be individually cultured from Day 4-Day 7. On Day 7, the embryo will be removed from the culture media it was grown in since day 4 (SCM). Both the embryo and the SCM will be frozen. The SCM may also be processed immediately rather than being frozen first. Embryos may be frozen starting at day 8 and through day 8.

There are multiple whole genome amplification (WGA) kits available commercially, some of which are specifically sensitive to low levels of DNA or fragmented DNA. The WGA kit through BioSkryb Genomics, “ResolveDNA”, has been shown successful previously in amplifying the cell-free DNA from SCM (See FIG. 11).

The amplified cell-free DNA from the spent culture media will then be subjected to array based genotyping along with the paired embryo (via biopsy). As the Illumina platform Next Generation Sequencing (FIG. 9) has been shown successful in producing sequence data from the cell-free DNA of SCM, it is reasonable to believe that the SNP array based genotyping panel should also lead usable genotypes.

To assess the quality of the genotype information produced from the cell-free DNA contained in the SCM, call rate and genetic concordance between these results and the biopsied genotype results of the paired embryo will be evaluated.

Imputation will be then performed on the genotypes of the cell-free DNA from the SCM, using a dataset containing parental genotypes and/or haplotypes as well as non-parental genotypes and/or haplotypes.

These imputed genotypes will then be used (as current bovine genotypes from somatic DNA are used) to estimate breeding values and expected progeny differences on economically relevant traits for selection purposes, and also in this case selecting which embryos to implant based on their genetic merit.

What is claimed is:

1. A method for genotyping an organism through cell-free DNA analysis from embryo culture media, comprising the steps of: collecting a sample of embryo culture media containing cell-free DNA; amplifying the cell-free DNA from the sample; and sequencing the amplified cell-free DNA to determine the genotype of the organism.

2. A device for genotyping an organism through cell-free DNA analysis from embryo culture media, comprising: a collection chamber for collecting a sample of embryo culture media containing cell-free DNA; an amplification unit for amplifying the cell-free DNA from the sample; and a sequencing unit for sequencing the cell-free DNA to determine the genotype of the organism.

3. A system for genotyping an organism through cell-free DNA analysis from embryo culture media, comprising: a collection device for collecting a sample of embryo culture media containing cell-free DNA; an amplification unit for amplifying the cell-free DNA from the sample; and a sequencing unit for sequencing the cell-free DNA to determine the genotype of the organism; wherein the system is capable of generating a genomic profile of the organism.

4. A computer-implemented method for genotyping an organism through cell-free DNA analysis from embryo culture media, comprising the steps of: collecting a sample of embryo culture media containing cell-free DNA; amplifying the cell-free DNA from the sample; sequencing the cell-free DNA; and comparing the sequence data to a reference genome to determine the genotype of the organism.

5. The device of claim 1, further comprising a computer processor for comparing the sequence data to a reference genome to determine the genotype of the organism.

6. A kit for genotyping an organism through cell-free DNA analysis from embryo culture media, comprising: a collection device for collecting a sample of embryo culture media containing cell-free DNA; an amplification unit for amplifying the cell-free DNA from the sample; a sequencing unit for sequencing the cell-free DNA; and a computer program for comparing the sequence data to a reference genome to determine the genotype of the organism.

7. The method of claim 1, further comprising comparing the sequence data to a reference genome to determine the genotype of the organism without causing harm to the organism.

8. The device of claim 2, further comprising a computer processor for comparing the sequence data to a reference genome to determine the genotype of the organism without causing harm to the organism.

9. The system of claim 3, further comprising a computer processor for comparing the DNA sequence to a reference genome to determine the genotype of the organism.

10. The method of claim 1, further comprising purifying the cell-free DNA from the sample.

11. The device of claim 2, further comprising a purification unit for purifying the cell-free DNA from the sample.

12. The system of claim 3, further comprising a purification unit for purifying the cell-free DNA from the sample; and a computer processor for comparing the DNA sequence to a reference genome to determine the genotype of the organism.

13. The method of claim 1, further comprising comparing the DNA sequence to a reference genome to determine the genotype of the organism.

14. The device of claim 2, further comprising a computer processor for comparing the sequence data to a reference genome to determine the genotype of the organism.

15. The system of claim 3, further comprising a computer processor for comparing the DNA sequence to a reference genome to determine the genotype of the organism.

16. The method of claim 1, further comprising comparing the DNA sequence to a reference genome to determine the genotype of the embryo.

17. The device of claim 8, wherein the device is for using cell-free DNA analysis from embryo culture media for prenatal genotyping.

18. The system of claim 15, wherein the system is utilized for using cell-free DNA analysis from embryo culture media for prenatal genotyping.

19. The method of claim 13, wherein the method is utilized for cell-free DNA analysis from embryo culture media for prenatal genotyping.

20. (canceled)

21. (canceled)

22. The method of claim 1, wherein the genotyping determines the sex of the organism.

23. The method of claim 4, wherein the method further comprises imputing the DNA sequence data.

24. A method for non-invasive embryo sexing of an organism through cell-free DNA analysis from embryo culture media. comprising the steps of: collecting a sample of embryo culture media containing cell-free DNA; amplifying the cell-free DNA from the sample; utilizing sex specific primers through PCR to bind to the cell-free DNA in the SCM, and determining the sex of the embryo.

25. A device for non-invasive embryo sexing of an organism through cell-free DNA analysis from embryo culture media. comprising: a collection chamber for collecting a sample of embryo culture media containing cell-free DNA; an amplification unit for amplifying the cell-free DNA from the sample utilizing sex specific primers through PCR to bind to the cell-free DNA in the SCM; and a computer processor to assess cell free-DNA amplification and primer binding to determine the sex of the embryo through the SCM.

26. A computer-implemented system for non-invasive embryo sexing of an organism through cell-free DNA analysis from embryo culture media, comprising: a collection chamber for collecting a sample of embryo culture media containing cell-free DNA; an amplification unit for amplifying the cell-free DNA from the sample utilizing sex specific primers through PCR to bind to the cell-free DNA in the SCM; and a computer processor to assess cell free-DNA amplification and primer binding to determine the sex of the embryo through the SCM.

27. A system for non-invasive embryo sexing of an organism through cell-free DNA analysis from embryo culture media, comprising the steps of: collecting a sample of embryo culture media containing cell-free DNA; amplifying the cell-free DNA from the sample; utilizing sex specific primers through PCR to bind to the cell-free DNA in the SCM, and determining the sex of the embryo.
